(19) United States
(12) Patent Application Publication
(10) Pub. No.: US 2012/0129809 A1
(43) Pub. Date: May 24, 2012

(54) LUNG CANCER TREATMENT

(75) Inventor: Scot Ebbinghaus, Lansdale, PA (US)

(73) Assignee: MERCK & CO.,, RAHWAY (NJ)

(21) Appl. No.: 13/138,474

(22) PCT Filed: Mar. 2, 2010
(86) PCT No.: PCT/US2010/000625

§ 371 Date: Feb. 8, 2012

Related U.S. Application Data
(60) Provisional application No. 61/209,102, filed on Mar. 2, 2009.

(51) Int. Cl. A61K 31/675; A61P 35/00; C07F 9/32

(52) U.S. Cl. 514/80; 546/23

(57) ABSTRACT

The invention provides a method for treating NSCLC, especially in cases of KRAS mutation, involving the administration of deforolimus.

### BACKGROUND OF THE INVENTION

Lung cancer is the most common cause of cancer death in the U.S. and worldwide. Approximately 215,020 new lung cancer cases are diagnosed in the U.S. each year, and estimated 1.44 million new lung cancer cases worldwide. Of patients who are diagnosed with lung cancer, more than 80% of patients eventually succumb to the disease. Histologically, the vast majority of patients with lung cancer have non-small cell lung cancer (NSCLC). Platinum doublet chemotherapy is the standard first-line treatment for NSCLC, and single agent chemotherapy or erlotinib provides clinical benefit in second-line patients. In spite of the advances in the treatment of NSCLC over the past decade, there remains a high unmet medical need for new treatments for lung cancer.

Recently, V-Ki-ras2 Kirsten rat sarcoma viral oncogene homolog (KRAS) mutations have been found to confer resistance to epidermal growth factor receptor (EGFR) targeted therapies in colorectal cancer. KRAS mutations are also observed in ˜25% of patients with NSCLC, and some studies have indicated that KRAS mutations are a negative prognostic factor in patients with NSCLC. Taken together, there is a need for new medical treatments for patients with NSCLC, especially those who have been diagnosed to have NSCLC character-ized by a KRAS mutation, and including those who have progressed after chemotherapy.

### SUMMARY OF THE INVENTION

Deforolimus is a unique analog of rapamycin that has demonstrated antiproliferative activity in a broad range of human tumor cell lines, including NSCLC, fibrosarcoma, glioblastoma, erythroleukemia, and prostate, colon, ovarian, endometrial and breast cancers. It has further demonstrated in vivo activity in murine tumor xenograft models utilizing human tumor cell lines representing glioblastoma, prostate, breast, lung, colon, and pancreatic cancers. In a large panel of more than 100 NSCLC cell lines, deforolimus activity was the same in both KRAS mutant and KRAS wild-type cells. Deforolimus showed anti-tumor activity in both KRAS mutant NSCLC xenografts (H2122 and A549) evaluated to date.

Deforolimus is currently in clinical development for the treatment of certain advanced cancers. It has shown evidence of anti-tumor activity in several tumor types, such as sarcomas, for which orally administered deforolimus is currently in a Phase III study. To date, however, there are no prior published reports evaluating deforolimus in clinical studies targeting NSCLC.

This invention provides a new approach for treating NSCLC patients, especially those whose NSCLC has been determined to be characterized by a mutation in KRAS, including among others patients who have responded to prior treatment and may be characterized with stable disease, those who have failed to respond or to respond adequately to prior treatment, those who may have responded to prior treatment but then experienced progression of the disease, and those who may have had such response followed by progression more than once.

One aspect of the invention involves administering to such a patient, e.g., a patient diagnosed with NSCLC characterized by a KRAS mutation, a treatment effective amount of deforolimus, e.g., on a schedule of daily administration for five consecutive days per week (“qd×5/7”), i.e., with a two day “holiday” between each 5-day course of treatment with deforolimus, typically over a period of multiple weeks, and in some cases indefinitely (e.g., until treatment is no longer necessary or tolerated).

In the practice of this invention, treatment effective amounts of deforolimus may be supplied to the patient using daily dosing levels of 2-160 mg on each of the five consecutive days per week, with doses of 10-60 mg being of particular current interest, especially doses from 20-40 mg. Deforolimus is typically taken after fasting (e.g., at least 2 hours after a light meal and 2 hours before eating).

A 40 mg dose of deforolimus administered orally on each of five consecutive days per week is of particular interest in the practice of this invention. The typical dose may be conveniently delivered with tablets containing 10 mg of deforolimus. Dosing may be briefly reduced or interrupted to manage side effects such as mouth sores. For example, in a 40 mg qd×5 regimen, the dose can be reduced to 10 mg for the remainder of the week, before resumption of the 40 mg regimen. As desired, the dose level may be increased in steps, e.g., to one or more intermediate levels for one or more weeks, before resumption of the full 40 mg dose in the typical regimen.

### DETAILED DESCRIPTION

### Deforolimus

The structure of deforolimus is depicted below:

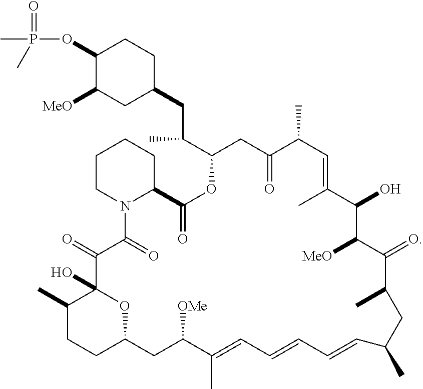

For further information on deforolimus, see e.g., U.S. Pat. Nos. 7,091,213 and 7,186,826, including Example 9 therein. Deforolimus has demonstrated antiproliferative activity in a variety of PTEN-deficient tumor cell lines, including glioblastoma, prostate, breast, pancreas, lung and colon (E. K. Rowinsky, Curr. Opin. Oncol., 2004, 16: 564-575). It has been designated as a fast-track product by the U.S. Food and Drug Administration for the treatment of soft-tissue and bone sarcomas and is currently in multiple clinical trials targeting certain hematologic malignancies and solid tumors.

A variety of oral and parenteral dosage forms are known for rapamycin and a number of rapamycin analogs (see e.g., U.S. Pat. No. 7,091,213) which may be used in the practice of this invention. Solid dosage forms are often of particular interest for oral administration and include among others conventional admixtures, solid dispersions and nanoparticles, typically in tablet, capsule, caplet, gel cap or other solid or partially solid form. Such formulations may optionally contain an enteric coating. Numerous materials and methods for such oral formulations are well known. See, e.g., US Patent Application US 2004/0077677 and Published International Patent Application WO04026280 (CCI-779). See also U.S. Pat. No. 6,197,781, U.S. Pat. No. 6,589,536, U.S. Pat. No. 6,555,132, U.S. Pat. No. 5,985,321, U.S. Pat. No. 6,565,859 and U.S. Pat. No. 5,932,243. For further background on deforolimus-containing tablets, for instance, see WO 2008/060546.

In addition to the foregoing, a wide variety of other methods and materials are also well known to those working in the field of macrolides like rapamycin and its derivatives. For additional background and examples of appropriate formulation technologies, see e.g., WO 03/064383 and US Published Patent Application 20050032825.

In practicing this invention, caution should be used when administering concomitant medications that induce, inhibit, or are metabolized by cytochrome P450 (CYP3A). Because deforolimus is extensively metabolized by CYP3A, the potential for drug-drug interactions, and the avoidance of such agents when possible, should be considered.

Caution should also be used when administering concomitant medications, such as warfarin, propranolol, phenytoin, and diazepam, which are extensively bound to plasma protein in case they might displace deforolimus from binding sites in plasma.

### EXAMPLES

The following examples describe approaches for practicing the invention. However, it should be understood that these examples are for illustrative purposes only and that the claims rather than the examples define the scope of the invention. Furthermore, unless the description in an Example is presented in the past tense, the text, like the rest of the specification, is not intended to suggest that experiments were actually performed or data were actually obtained. All documents referred to in the Examples and elsewhere in this document are incorporated herein in their entirety.

Deforolimus

Deforolimus may be prepared as described in U.S. Pat. No. 7,091,213 and supplied as enteric coated tablets containing 10 mg drug/tablet, prepared as described in WO 2008/060546 and dispensed on a blister card. The tablets or other pharmaceutical composition containing the drug may be supplied in a kit further containing instructions for their administration to patients diagnosed with NSCLC characterized by a KRAS mutation.

KRAS Mutation Testing

KRAS mutations, e.g., at codons 12 or 13, may be detected from pathology samples taken from the patient. In one approach to such testing, DNA is removed from the sample and tested against labeled oligonucliotide probes using PCR to amplify the targeted mutated DNA to permit detection. In some countries, commercial testing centers carry out such tests. Alternatively, a test kit such as the TheraScreen: K-RAS Mutation kit (DxS Ltd, 48 Grafton Street, Manchester M13 9XX, UK) may be used. The TheraScreen kit can detect mutations in codons 12 and 13 of the KRAS oncogene:

| | |
|---|---|
| Gly12Asp (GGT > GAT) | Gly12Arg (GGT > CGT) |
| Gly12Ala (GGT > GCT) | Gly12Cys (GGT > TGT) |
| Gly12Val (GGT > GTT) | Gly13Asp (GGC > GAC) |
| Gly12Ser (GGT > AGT) | |

For further information on the TheraScreen kit, its use and the underlying biology, the reader is directed to the supplier's website:

<http://www.dxsdiagnostics.com/Content/TheraScreenKRAS.aspx>

and to the following references:
E. Massarelli, M. Varella-Garcia, X. Tang, A. C. Xavier et al. (2007). KRAS Mutation Is an Important Predictor of Resistance to Therapy with Epidermal Growth Factor Receptor Tyrosine Kinase Inhibitors in Non-Small Cell Lung Cancer. Clin Cancer Res 2007; 13 (10).
William Pao, Theresa Y. Wang, Gregory J. Riely, Vincent A. Miller, Qiulu Pan, Marc Ladanyi et al. (2005). KRAS mutations and Primary Resistance of Lung Adenocarcinomas to Gefitinib or Erlotinib. PloS Medicine 2(1): 57-61.
D. A. Eberhard, B. E. Johnson, L. C. Amler, A. D. Goddard et al (2005). Mutations in the EGFR and in K-RAS are predictive and prognostic indicators in patients with NSCLC treated with chemotherapy alone and in combination with erlotinib. J Clin Oncol 23: 5900-5909.
L. Toschi & F. Cappuzzo. (2007) Understanding the new genetics of responsiveness to EGFR tyrosine kinase inhibitors. Oncologist 12; 211-220.
Sae-Won Han, Tae-You Kim, Yoon Kyung Jeon, Pil Gyu Hwang et al. (2006). Optimization of Patient Selection for Gefitinib in Non-Small Cell Lung Cancer by combined analysis of Epidermal Growth Factor Receptor Mutation, K-RAS Mutation, and AKT Phosphorylation. Clin Cancer Res 12(8):2538-2544.
Newton C R, Graham A, Heptinstall L E, Powell S J, Summers C et al. (1989). Analysis of any point mutation in DNA. The amplification refractory mutation system (ARMS) Nucleic Acids Res. 17 (7): 2503-16.
R. G. Amado et al. (2007) Analysis of K-RAS mutations in patients with metastatic colorectal cancer receiving panitumumab monotherapy. Presented at ECCO 2007.
C. Bokemeyer et al., K-RAS status and efficacy of first-line treatment of patients with metastatic colorectal cancer (mCRC) with FOLFOX with or without cetuximab: The OPUS experience. J Clin Oncol 26: 2008 (May 20 suppl; abstr 4000).
E. Van Cutsem et al., K-RAS status and efficacy in the first-line treatment of patients with metastatic colorectal cancer (mCRC) treated with FOLFIRI with or without cetuximab: The CRYSTAL experience. J Clin Oncol 26: 2008 (May 20 suppl; abstr 2).
S. Tejpar et al., Relationship of efficacy with K-RAS status (wild type versus mutant) in patients with irinotecan-refractory metastatic colorectal cancer (mCRC), treated with irinotecan (q2w) and escalating doses of cetuximab (q1w): The EVEREST experience (preliminary data). J Clin Oncol 26: 2008 (May 20 suppl; abstr 4001).

### Example 1

### Treatment with Deforolimus (40 mg, p.o., qd×5)

NSCLC patients to be treated in this example have already been determined to have a KRAS mutation.

A 40 mg dose of deforolimus is self-administered orally, in the form of four 10 mg enteric coated tablets, each day for 5 consecutive days each week. Deforolimus should be taken with water 2 hours after a light meal (i.e.: toast, tea, etc.). Patients may be instructed to consume only water for 2 hours after dosing with the deforolimus.

During the course of treatment, patients should consult their care giver before taking any strong inducers or inhibitors of CYP3A and before consuming grapefruit or grapefruit juice.

Progression, spread or remission of the cancer and the condition of the patient may be followed by periodic monitoring of one or more indicators, such as Prostate Specific Antigen level, bone scan, CT scan of abdomen and pelvis, levels of circulating tumor cells, etc.

### Example 2

### General Dosing Modification Guidance

General guidance for dosage modification for the majority of adverse drug reactions is provided in the table below. This table outlines some recommended dose modification steps in the event a patient has a ≧Grade 2 adverse event (other than mouth sores or pneumonitis) believed to be related to the administration of deforolimus. Occurrence refers to a specific, repeating adverse event. That is, “second” means the second episode of the event following resolution of the first episode to ≦Grade 1. For purposes of the example, it is assumed that patients are taking the drug Monday through Friday.

General Dosage Modification

| | Action | Sat/ | | Sat/ | Action during second week of or |
|---|---|---|---|---|---|
| Occurrence | Until Friday | Sun | Action during next week | Sun | more |
| First | Decrease | No | If AE ≦Grade 1 (resolved) on | No | If AE resolved on Monday may |
| | to 10 mg (1 | Drug | Monday may increase dose to 40 | Drug | increase dose to 40 mg; otherwise |
| | tab) | | mg (4 tabs); Otherwise continue | | stop drug until resolution and resume |
| | | | at 10 mg through Friday | | 40 mg |
| Second | Decrease | No | If AE resolved on Monday may | No | If AE resolved on Monday may |
| | to 10 mg (1 | Drug | increase dose to 30 mg for | Drug | increase dose to 30 mg for remainder |
| | tab) | | remainder of study; otherwise | | of study; otherwise stop drug until |
| | | | continue at 10 mg through Friday | | resolution and resume 30 mg |
| Third | Decrease | No | If AE resolved on Monday may | No | If AE resolved on Monday may |
| | to 10 mg (1 | Drug | increase dose to 20 mg for | Drug | increase dose to 20 mg for remainder |
| | tab) | | remainder of study; otherwise | | of study; otherwise stop drug until |
| | | | continue at 10 mg through Friday | | resolution and resume 20 mg |
| Fourth | Decrease | No | Continue drug at 10 mg through | No | If AE resolved on Monday may |
| | to 10 mg (1 | Drug | Friday | Drug | continue on drug at 10 mg; otherwise |
| | tab) | | | | stop drug until resolution and resume |
| | | | | | at 10 mg or consider withdrawal of |
| | | | | | patient |

### Example 3

### Mouth Sores and Dosing Modification Guidance

A common side effect associated with deforolimus is the occurrence of mouth sores typically reported as mucositis. The sores associated with deforolimus are distinct ulcers that most closely resemble aphtous ulcers. They are usually painful and can be up to 1 cm in widest diameter. The onset of such mouth sores may occur as early as during the first week of treatment deforolimus and usually resolves during regularly scheduled treatment holidays or following dose reductions and/or delays.

Treatment of mouth sores should include dose modification as described in the table in the previous example, as well as palliative pain management with the type and strength of the analgesia escalating in parallel with the severity of the mouth sore pain. Topical analgesics may be employed if felt to be beneficial. The following treatment plan is suggested:
Bicarbonate rinses 4 times a day every day if there are any oral mucosal symptoms or signs. (There do not have to be actual ulcers to institute this prophylactic measure.)
At the appearance of mouth sores, use topical analgesics such as Orajel® Medicated Mouth Sore Swabs, or equivalent, as needed to achieve pain relief and allow normal eating.
Use other agents such as Gelclair® or anesthetic mouth washes only if dose reduction and application of topical analgesics do not result in pain control.

Mouth Sores Dosage Modifications

| NCI CTCAE | |
|---|---|
| Grade* | Dose Delay and or Reduction Required |
| Grade 1 | Continue treatment without dose interruption or reduction. |
| | Begin symptomatic/prophylaxis treatment. |
| Grade 2 | First Episode: |
| or higher | Reduce dose to 10 mg per day for remainder of week. |
| | Resume 40 mg per day after the two-day break if |
| | improvement to Grade 1 or less |
| | If Grade 2 or higher, dose 10 mg per day for additional week |
| | Second Episode: |
| | Reduce dose to 10 mg per day for remainder of week. |
| | Resume 30 mg per day after the two-day break if |
| | improvement to Grade 1 or less |
| | If Grade 2 or higher, dose 10 mg per day for additional week |
| | Third Episode: |
| | Reduce dose to 10 mg per day for remainder of week. |
| | Resume 20 mg per day after the two-day break if |
| | improvement to Grade 1 or less |
| | If Grade 2 or higher, dose 10 mg per day for additional week |
| | Fourth episode or greater: |
| | 1. Stop treatment until improved to ≦Grade 1 |
| | 2. Improvement occurs in ≦2 weeks |
| | Resume at 10 mg per day |
| | 3. Improvement occurs in >2 weeks |
| | Contact Medical Monitor and consider permanently |
| | discontinuing study drug |

NOTE:
Mouth sores refers to following terms: aphthous stomatitis, gingival pain, gingival ulceration, glossitis, mouth ulceration, oral discomfort, oral pain, stomatitis, and mucosal inflammation.
*NCI CTCAE Grade based on functional/sympotomatic criteria described for mucositis/stomatitis

### Example 4

### Additional Safety Guidance

(a) pneumonitis

In cases of symptomatic pneumonitis, the care giver should consider immediate cessation of deforolimus treatment and evaluation of the patient to rule out other causes such as infection. If the patient is diagnosed with drug-related pneumonitis, the following treatment plan is currently recommended.

Recommended treatment for symptomatic pneumonitis:
Dose interruption and steroid intervention for ≦Grade 2 with option to return to treatment if improves to Grade 1 or resolves within 4 weeks
Patients should begin a regimen of steroids (e.g., prednisone 60-80 mg daily 1-2 weeks) tapering over 1-4 weeks. Deforolimus treatment may be resumed once clinical improvement is observed
Immediate discontinuation if ≧Grade 3.

After improvement to ≦Grade 1 of the pneumonitis the following rules should apply:
First episode of pneumonitis
Improvement occurs in ≦2 weeks—Resume full dose
Improvement occurs in >2 weeks—Resume at 10 mg lower than starting dose
Second episode of pneumonitis
Permanently discontinue deforolimus treatment if upon study drug rechallenge patient develops pneumonitis ≧Grade 2.

Patients who are asymptomatic but have findings of pneumonitis should stop deforolimus treatment for one week and during that week receive steroids (e.g., 60 mg prednisone). If there is no improvement in the signs of pneumonitis, additional diagnostic procedures should be considered, such as bronchoscopy, to confirm the diagnosis. If there is improvement in the pneumonitis, the patient may resume deforolimus treatment while undergoing a 1-2 week taper of the steroids. The patient should be followed every 2-4 months by chest x-ray.

### (b) Hypertriglyceridemia/Hypercholesterolemia

Hyperlipidemia is a common adverse reaction associated with rapamycin analogs. Clinically significant elevations above baseline levels of cholesterol and/or triglyceride levels should be immediately managed with respect to the patient's overall condition; both statins and fibrate agents have been used in patients receiving deforolimus. No dose interruptions or reductions in deforolimus is usually necessary.

1. A method for treating non-small cell lung cancer (NSCLC) in a patient in need thereof, wherein the NSCLC is characterized by a KRAS mutation, the method comprising administering to the patient a treatment effective amount of deforolimus.

2. A method for treating non-small cell lung cancer (NSCLC) in a patient diagnosed with NSCLC characterized by a KRAS mutation, the method comprising administering to the patient a treatment effective amount of deforolimus.

3. A method for treating non-small cell lung cancer (NSCLC) in a patient, the method comprising (i) determining whether the patient has NSCLC characterized by a KRAS mutation; and (ii) if the patient has NSCLC characterized by a KRAS mutation, administering to the patient a treatment effective amount of deforolimus.

4. The method of any of claims 1-3, wherein the deforolimus is administered orally.

5. The method of any of claims 1-4, wherein the deforolimus is administered orally in a dose of 2-160 mg/day for five consecutive days per week.

6. The method of claim 5, wherein the deforolimus is administered in a dose of 20-40 mg/day for five consecutive days per week.

7. The method of claim 3, wherein if the patient has NSCLC that is not characterized by a KRAS mutation, then no deforolimus is administered to the patient.

8. The use of deforolimus in the preparation of a medicament for oral administration for the treatment of NSCLC characterized by a KRAS mutation.

9. A kit comprising (i) a pharmaceutical composition comprising deforolimus; and (ii) instructions for administering the pharmaceutical composition to a patient diagnosed with NSCLC characterized by a KRAS mutation.
